(19) United States
(12) Patent Application Publication
(10) Pub. No.: US 2023/0277717 A1
(43) Pub. Date: Sep. 7, 2023

(54) UV-C LIGHT IN THE VENTILATOR UNIT OF INDIVIDUALLY VENTILATED CAGING SYSTEM

(71) Applicants: Parag Vijay JAIN, Nashik, Maharashtra (IN); Dhara Parag JAIN, Nashik, Maharashtra (IN)

(72) Inventors: Parag Vijay JAIN, Nashik (IN); Dhara Parag JAIN, Nashik (IN)

(21) Appl. No.: 18/019,323

(22) PCT Filed: Jul. 27, 2021
(86) PCT No.: PCT/IN2021/050728

§ 371 Date: Feb. 2, 2023

(30) Foreign Application Priority Data
Aug. 6, 2020 (IN) 202021023834

(51) Int. Cl. A61L 9/22; A61L 9/20; A01K 1/00; A01K 1/03; B01D 46/00; B01D 46/62

(52) U.S. Cl. CPC A61L 9/22; A61L 9/20; A01K 1/0047; A01K 1/03; B01D 46/0028; B01D 46/0043; B01D 46/0049; B01D 46/62; A61L 2209/14; A61L 2209/16; A61L 2209/12; A61L 2209/111; A01K 1/031; B01D 2279/65; B01D 2273/30; B01D 2279/35

(57) ABSTRACT

This invention claims Individually Ventilated Caging system (IVC system) for small animals comprising a Ventilation Unit (1) comprising an initial supply chamber (12) for taking external air inside the system for filtering and supplying to the cages in IVC system and an exhaust chamber (22) for exhausting the used air collected from the cages in the IVC system; the supply chamber and the exhaust chamber provided with filter/s (13),(14), (17), (18) for the incoming or outgoing/exhausting air; wherein the IVC system is characterized by devices additional to the filters for disinfecting incoming air and/or disinfecting out-going air exhausted from the cages. The additional devices comprise (a) UVC light (15), and/or (b) one or more electrostatic air filter/s and/or (3) an ionizer on supply and or exhaust side.

### FIELD OF INVENTION

The present invention relates to the incorporation of UV-C light as additional source of protection against viruses not being filtered by supply and or exhaust HEPA (High-Efficiency Particulate Air) or ULPA (Ultra-Low Particulate Air) filters of the ventilator unit of IVC (Individually Ventilated Caging) system used for small animals like mice or guinea pigs.

### BACKGROUND OF THE INVENTION

Ultraviolet (UV) light is a form of light that is invisible to the human eye. It occupies the portion of the electromagnetic spectrum between X-rays and visible light. The sun emits ultraviolet light; however, much of it is absorbed by the earth's ozone layer.¹

UV light has three wavelength categories: UV-A, UV-B, and UV-C.

UV-C part of the ultraviolet light spectrum emits a high frequency of UV light that makes it extremely effective at killing bacteria, viruses, mold and other pathogens. Killing bacteria with UV light requires the use of germicidal wavelengths of 185-254 nanometers (nm).²

“Sanitizing with UV-C light has been a normal practice since the mid-20th century. In fact, the 1903 Nobel Prize in Medicine was awarded to Niels Finsen for using UV light to fight tuberculosis. Today, hospitals and laboratories use UV light to keep their facilities sterile, which means it perfect for sanitizing mobile devices, which are especially good at harboring and growing bacteria.”³

Viruses are obligate parasites that are biologically active only within their host. Viruses can be transmitted by various routes, including direct and indirect contact, vector transmission, and vehicle transmission. For deadly viruses such as Severe Acute Respiratory Syndrome (SARS) virus, influenza virus, and enterovirus, the vehicle transmission pathways include respiratory transmission by droplets and aerosols, as well as fecal-oral transmission via water, food, and environmental surfaces. To reduce infection risk from virus infection, control techniques for inactivating such viruses have been extensively researched (Jensen 1964; Gerba et al. 2002; Shin et al. 2003; Thurston-Enriquez et al. 2003). Among these control techniques, ultraviolet germicidal irradiation (UVGI) was demonstrated to be extremely efficient for virus inactivation (Jensen 1964; Galasso et al. 1965; Gerba et al. 2002; Nuanualsuwan et al. 2003; Thurston-Enriquez et al. 2003).The mechanisms of UVGI on microbes are uniquely vulnerable to light at wavelengths at or near 253.7 nm, because the maximum absorption wavelength of aDNA molecule is 260 nm. The pyrimidine of DNA base can strongly absorb UV light. After irradiation, the DNA sequence where pyrimidine and pyrimidine link can form pyrimidine dimers. These dimers can change the DNA double helix structure and interfere with DNA duplication, as well as lead to the destruction of the replicate ability of cells and thus render the cells non-infectious (Brickner et al. 2003).⁴

In the field of laboratory animal management, in particular with small animals such as mice or guinea pigs, cages are used in particular sealed cage suitable to be connected to a ventilation system. Cages are usually housed side by side and stacked on shelves or stacked in the cabinet constructed for this purpose. Environment inside these cages must be tightly controlled to prevent contamination of the animals by the external environment and/or contamination of the environment and humans by the animals. Individually Ventilated Caging (IVC) systems are widely used for housing laboratory animals that enable a plurality of such animal cages to be arranged in an industrious and efficient manner. These systems are designed for providing a highly consistent environment across all cages.

Prior art IVC system comprises of Ventilat or unit for air supply and exhaust or for exhaust only; an IVC Cage or cages and Racks or cabinets to hold the IVC Cage or Cages. Ventilator unit works to supply High-Efficiency Particulate Air (HEPA) or Ultra-Low Particulate filtered Air (ULPA) using the fan or blower to the IVC Cage and exhaust air from the IVC Cage through the distribution network of the rack/cabinet connections. In an alternative system, also known as EVC (Exhaust Ventilated Caging System), the cages take in air directly from the room in which they are held in racks, the Ventilator unit in such cases works only to exhaust the air from the cages to outside environment.

Each ventilator unit is fitted with blower or fan, pre-filters and HEPA or ULPA filters on supply and or exhaust side so that HEPA or UPLA filtered air can be supplied to the IVC Cage and or exhausted air from the IVC Cage can be HEPA or ULPA filtered before releasing to the external environment.

HEPA filters can capture at least 99.97% of particles with a diameter greater than or equal to 0.3 μm. Ultra-low particulate (or sometimes “penetration”) air (ULPA) filters are closely related to HEPA filters but are even more efficient in their filtration capability. ULPA filters are specified to remove 99.999% of contaminants 0.12 μm or larger in diameter.

Thus the Ventilator unit of IVC Caging System takes in ambient air and supplies HEPA or ULPA filtered air to the IVC Cage and or exhaust the air inside the IVC Cage and release the HEPA or ULPA filtered air to the external environment.

The purpose of the ventilator unit is to provide filtered air free from viruses, allergens, bacteria or pathogens so as to protect the animal inside IVC Cage and also if any infection model is created in the animal within the IVC Cage then release the exhausted air from the IVC cage by proper filtration so that no viruses, allergens, bacteria or pathogens are released in the external environment.

Although HEPA filters are high in efficiency, yet they have efficiency for size range 0.1 to 0.3 micron only. Many viruses have size in the range of 0.004 to 0.3 μ which would make access though the filtered air of Ventilator unit of IVC Caging system to the IVC Cage leading to a potential threat to the animal in the IVC Cage or release of such virus from IVC Cage to the external environment during the exhaust creating potential threat to the humans.

Hence, there was a need of improved individually ventilated caging system.

### SUMMARY OF INVENTION

This invention claims Individually Ventilated Caging system (IVC system) for small animals comprising a Ventilation Unit 1 comprising an initial supply chamber 12 for taking external air inside the system for filtering and supplying to the cages in IVC system and an exhaust chamber 22 for exhausting the used air collected from the cages in the IVC system; the supply chamber and the exhaust chamber provided with filter/s 13,14, 17, 18 for the incoming or outgoing/exhausting air; wherein the IVC system is characterized by devices additional to the filters for disinfecting incoming air and/or disinfecting out-going air exhausted from the cages. The devices additional to the filters for disinfecting incoming air and/or disinfecting out-going air exhausted from the cages comprises (a) UV C light 15, and/or (b) one or more electrostatic air filter/s and/or (3) an ionizer on supply and or exhaust side.

The IVC system according to this invention comprises a Ventilator unit 1 which has supply section and/or exhaust section, HEPA or ULPA filters provided in supply section and exhaust section for filtering air passing through these sections to supply the UV-C light 15 treated filtered air through supply ports 3 and 4 to the IVC Cages placed on the Rack 10 through a supply distribution network 8 connected to ventilator unit 1 through a suitable flexible or non flexible pipe 30a.

The IVC system according to this invention also comprises an exhaust distribution network 9 of the rack for collecting and return of the used air from the cages back to the Ventilator unit 1 through an exhaust port 6 of the Ventilator Unit 1, the exhaust distribution network 9, the exhaust port being interconnected with the exhaust distribution network 9 through a flexible or non flexible pipe 30b, the exhaust air brought back to Ventilation Unit 1 being treated with UVC light 19 and being filtered through HEPA- or ULPA-filtration; and return to the external environment though exhaust port 7 of the Ventilator unit 1.

The Ventilator unit 1 comprises a Control Panel with display 2 where different operational parameters are displayed.

The IVC system also comprises one or more Racks 10 for holding IVC Cages are provided each being supplied with the UV-C light 15 treated filtered air through additional supply ports. Parameters displayed on the Control Panel consist of one or more selected from the group consisting of air changes per hour, positive or negative pressure modes, number of cages, temperature, relative humidity, ammonia and other environmental parameters.

The Ventilator unit 1 has upper half as a supply section and lower half as exhaust section. The supply section comprises one or more supply blower or fan 12, duct of supply blower 23, supply side HEPA or supply side ULPA filter 13, supply side pre-filter 14, one or more UV-C lights 15, initial supply chamber 21 and final supply chamber 25. The exhaust section comprises one or more exhaust blower or fan 16, duct of exhaust blower 24, exhaust side HEPA or exhaust side ULPA filter 17, exhaust side pre-filter 18, one or more UV-C light 19 and initial exhaust chamber 22.

The Ventilator unit 1 has filtered air supply ports on supply side labeled as supply port no. 3 and 4, each one connecting to separate rack units 10 for holding the IVC Cage in the supply distribution Network 8; and exhaust air outlet ports on exhaust side labeled as exhaust port no. 5 and 6 coming from two separate rack units 10 for connecting with the exhaust distribution network 9 of one or more rack for holding the IVC Cages 11.

The Ventilator unit 1 comprises an air inlet 20 for taking external air in for filtration and circulation. Supply blower or fan 12 takes the air from the external environment through the air inlet 20 for filtration, treatment with UV-C light 15 and supplies to the IVC Cages. The initial supply chamber 21 is fitted with one or more UV-C light 15 either above the filters or below the prefilter or between pre-filter 14 and HEPA filter or ULPA filter 13 or at any two locations mentioned above or at all the three locations mentioned herein,

The Ventilator unit 1 also comprises an air supply port on supply side as supply port nos. 3 and 4 for connecting with the rack 10 that holds the IVC Cages. The initial exhaust chamber 22 is fitted with one or more UV-C light 19. The UV-C light 19 may be fitted before or after the filter or between pre-filter 18 and HEPA filter or ULPA filter 17 or at any two locations mentioned above or at all the three locations mentioned herein. In the initial exhaust chamber 22 the air is passed through supply pre-filter 14 and supply side HEPA or supply side ULPA filter 13 for further removing the particles in the air stream. This filtered air then passes through the duct of supply blower 23 and through the supply blower or fan 12 and then to the Final supply chamber 25. The filtered air in the final supply chamber 25 then passes through the supply ports 3 and or 4 or more ports in case more racks are connected to the ventilator Unit 1 to the supply distribution network 8 of the rack 10 for holding IVC Cage and eventually to the IVC Cages 11 loaded on the rack 10. The air from the cage is exhausted, filtered and treated with UV-C light in the exhaust section of the ventilator unit 1 for disinfection and the treated air is released to the external environment from the outlet port 7.

The IVC system as claimed in claim 5 wherein the wattage of UV-C 15 and UV-C 19 lights is selected to provide disinfection of the volume of air that is drawn in respective supply chamber and exhaust chamber and within residence time of the air within these chambers.

Each IVC Cage 11 has one cage air supply valve 27 and/or cage air exhaust valve 26 situated either on cage bottom 28 or alternatively they can also be situated on cage lid 29. Cage lid 29 and cage bottom 28 are connected together so as to form airtight connection.

Air from the rack for holding IVC Cage 10, passes through the supply distribution network 8 to the cage air supply valve 27 of the IVC Cage 11, filtered and UV-C light treated air then reaches inside the IVC Cage 11 providing ventilation for the animals within the IVC Cage 11. Air inside the IVC Cage 11 is then exhausted from the cage air exhaust valve 26 using the Exhaust distribution network 9 to the Exhaust port 5 and or 6 of the Ventilator 1 due to the suction created by Exhaust blower or fan 16.

The exhausted air first reaches for treatment and filtration to initial exhaust chamber 22 fitted with one or more UV-C light 19, pre-filter 18 and exhaust side HEPA or exhaust side ULPA filter 17. This air after treatment with UV-C light 19 and filters and then passes through the duct of exhaust blower 24, to fan 16 and exhaust port 7 and then eventually released to the external environment by suitable means.

The IVC system is designed for small animals which comprise, without limitation, mice, guinea pigs and the like.

### DETAILED DESCRIPTION OF THE INVENTION

A significant body of scientific research like Bintsis et al. 2000⁵, Yaun et al. 2004⁶, Do-Kyun Kim et al 2018⁷, Lyndon 1977⁸, Walker et al 2007⁹ has proven UV-C light's ability to inactivate an extensive list of pathogenic bacteria, viruses and protozoa.

In one aspect this invention relates to Individually Ventilated Caging system (IVC) for small animals characterized by providing devices additional to the filters for disinfecting incoming air and/or disinfecting out-going air exhausted from the cages. The devices additional to the filters for disinfecting incoming air and/or disinfecting out-going air exhausted from the cages comprises (a) UV C light 15, and/or (b) one or more electrostatic air filter/s and/or (3) an ionizer on supply and or exhaust side. The UV-C light may be fitted either above the filters or below the prefilter or between pre-filter 14/18 and HEPA filter or ULPA filter 13/17 or at any two locations mentioned above or at all the three locations mentioned herein.

This feature provides additional protection to the caged animal by killing the bacteria and/or viruses in the air before supplying to the IVC Cage. In another aspect of this invention, providing UV-C light on the exhaust side of HEPA or ULPA filter provides additional protection to the external environment by killing the bacteria and/or viruses in the exhausted air from the cage before releasing to the external environment.

Addition of UV-C Light source in supply and or exhaust side also provide additional protection to the ventilator's inside area by killing the bacteria, allergens or viruses already gathered on sor other nearby areas within the range of UV-C light source..

### BRIEF DESCRIPTION OF FIGURES AND LEGENDS

FIG. 1 schematically shows complete individually ventilated caging (IVC) system comprising Ventilator unit 1, Rack 10 for holding IVC Cages, IVC Cage 11. UVC light treated, HEPA or ULPA filtered air is supplied by Ventilator unit to the IVC Cages placed on the Rack for holding IVC Cages through the Supply port 3 of the Ventilator unit 1 and Supply distribution network 8 of the rack which are interconnected through suitable flexible or non flexible pipe 30a. Similarly, the air from the cage is returned through the Exhaust distribution network 9 of the rack and exhaust port 6 of the Ventilator which are inter connected through suitable flexible or non flexible pipe 30b back to the Ventilator unit 1 where it is treated with UVC light either before and or after HEPA or ULPA filtration; and is returned to the external environment though exhaust port 7 (shown in FIG. 2) of the Ventilator unit 1. More similar racks can be connected to same Ventilator unit 1 depending on the blower capacity. As an illustration one more rack is shown connected to same Ventilator unit 1 in FIG. 1. For each such additional rack 10 that may be connected to Ventilator unit 1, system features are replicated mutatis mutandis for such additional racks. Ventilator unit 1 has a Control panel with display 2 where different parameters like air changes per hour, pressure mode etc. can be displayed.

FIGS. 2(a) and 2(b) schematically shows cutaway view of Ventilator unit 1.

Ventilator unit 1 has supply section and/or exhaust section. Supply section comprises one or more Supply blower or fan 12, Duct of supply blower 23, supply side HEPA or supply side ULPA filter 13, supply side pre-filter 14, one or more UV-C lights 15, Initial supply chamber 21 and Final supply chamber 25. Similarly exhaust section comprises one or more Exhaust blower or fan 16, Duct of exhaust blower 24, Exhaust HEPA or ULPA filter 17, Exhaust pre-filter 18, one or more UV-C light 19 and Initial exhaust chamber 22.

FIG. 3 shows schematic diagram of Ventilator unit showing the air inlet 20 for taking external air: Ventilator unit (1) has air inlet 20 for taking external air in for filtration and circulation. Supply blower or fan 12, shown in FIG. 2(a) and FIG. 2(b), takes the air from the external environment through the air inlet 20 and this air is further processed for filtration and supplied to the IVC Cages.

FIG. 4 shows top view of Ventilator unit showing supply port 3 and 4 and exhaust port 7: Ventilator unit 1 has air supply port on supply side as Supply port no. 3 and 4 for connecting with the Rack 10 for Holding the IVC Cage . Air from the cage is exhausted and filtered in the exhaust section of the Ventilator unit 1 and the filtered air is released to the external environment from the outlet port 7.

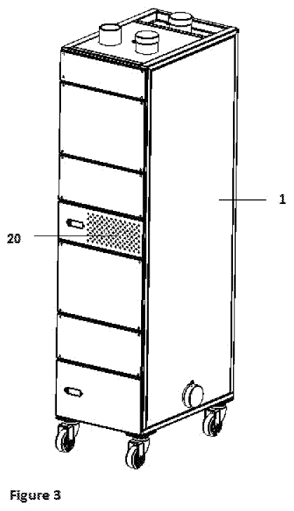

FIG. 5 Schematic of IVC Cage 11

Each IVC Cage 11 has one Cage air supply valve 27 and/or Cage air exhaust valve 26 situated either on Cage bottom 28 or alternatively they can also be situated on Cage lid 29. Cage lid 29 and cage bottom 28 are connected together so as to form airtight connection.

To each Ventilator unit 1, depending on the capacity of the blower, more than one units of racks 10 can be attached and each one being supplied with filtered air supplying line and or exhaust line. Ventilator unit 1 has air supply port on supply side as Supply port no. 3 and 4, or more depending on number of racks attached to the Ventilator unit 1, for connecting with the Supply Distribution Network 8 for one or more Rack 10. Similarly, Ventilator unit 1 has exhaust air outlet ports on exhaust side as Exhaust port no. 5 and 6 for connecting with the Exhaust Distribution Network 9 for one or more Rack/s 10 for Holding the IVC Cage.

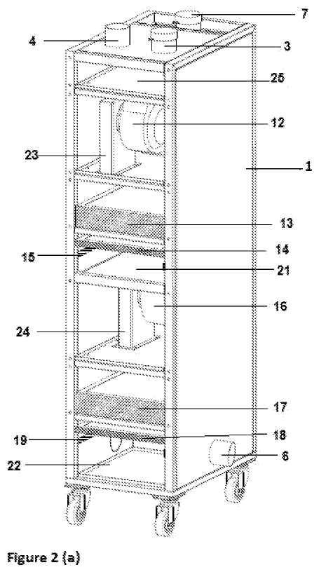

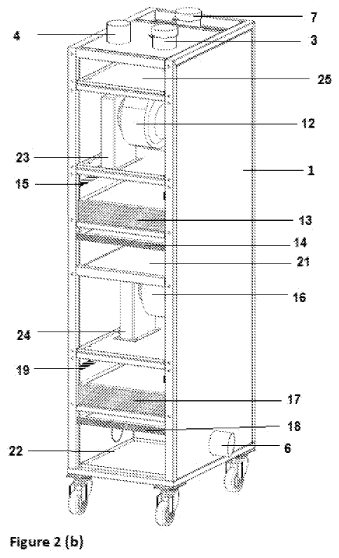

Ventilator unit 1 has air inlet 20 (FIG. 3) for taking external air in for filtration and circulation. Supply blower or fan 12 (FIG. 2) takes the air from the external environment through the air inlet 20 and this air enters in the Initial supply chamber 21. Initial supply chamber 21 is fitted with one or more UV-C light 15 either above the filters or below the prefilter or between pre-filter 14 and HEPA filter or ULPA filter 13 or at any two locations mentioned above or at all the three locations mentioned herein. The wattage of UV-C 15 light shall differ from location to location depending upon the microbial load of samples of input air that is drawn in and of the outgoing air and adjusting the wattage to a point wherein 100% preferably or a lower level that is considered as acceptable for sterility/disinfection of the outcoming air is attained.

In one embodiment of the Ventilator unit, the air flow rate on the supply side or exhaust side is not more than 0.9 cfm whereas the intensity of 254 nm UV radiation from the two light sources of 12 W light was found to be more than 1450 μW/cm². The UV-C light 15 acts as germicidal lamp and kills the bacteria, viruses, mold and other pathogens in the stream of the air taken in by the Supply blower or fan 12. This air is treated with UV-C light 15 either before or after filtration or at both instances. Filtration may be done in many different ways. Most convenient method is by passing through supply side Pre-filter 14 and supply side HEPA or ULPA filter 13 for further removing the particles in the air stream. This filtered air then passes through the Duct of supply blower 23 and through the Supply blower or fan 12 and then to the Final supply chamber 25. Filtered air in the Final supply chamber 25 then passes through the Supply port 3 and or 4 to the Supply distribution network 8 of the Rack 10 for holding IVC Cage and eventually to the IVC Cages 11 loaded on the Rack 10. The UV C light can be fitted at any position below or above or at both locations with respect to HEPA/ULPA filter/s of the supply section.

Ventilator unit 1 (FIG. 2) has supply section (upper half) and exhaust section (lower half). Supply section comprises one or more Supply blower or fan 12, Duct of supply blower 23, one or more supply side HEPA or ULPA filter of same or different types 13, one or more supply side pre-filter of same of different types including active carbon filters for odor removal 14, one or more UV-C light 15, Initial supply chamber 21 (supplied with incoming air from environment through air inlet 20) and Final supply chamber 25. Similarly exhaust section comprises one or more Exhaust blower or fan 16, duct of exhaust blower 24, one or more Exhaust HEPA or ULPA filter of same or different types 17, one or more Exhaust pre-filter 18 of same or different types including active carbon filters for odor removal, one or more UV-C light 19 either below or above the filter or at both places and Initial exhaust chamber 22.

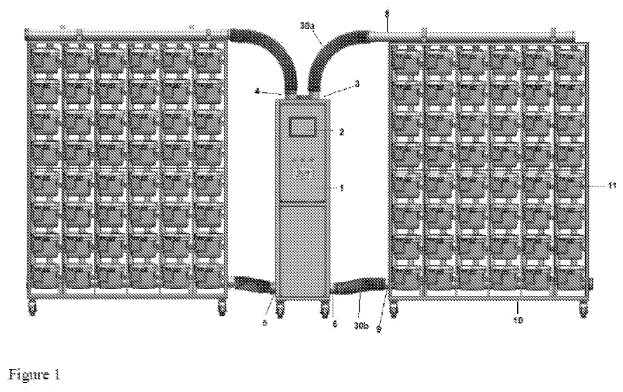

Ventilator unit 1 has filtered air supply ports on supply side labeled as supply port no. 3 and 4, each one connecting to separate rack units 10 for holding the IVC Cage in the Supply Distribution Network 8. Similarly, ventilator unit 1 has exhaust air outlet ports on exhaust side labeled as exhaust port no. 5 and 6 (shown in FIG. 1) coming from two separate rack units for connecting with the exhaust distribution network 9 of one or more rack for Holding the IVC Cage 10.

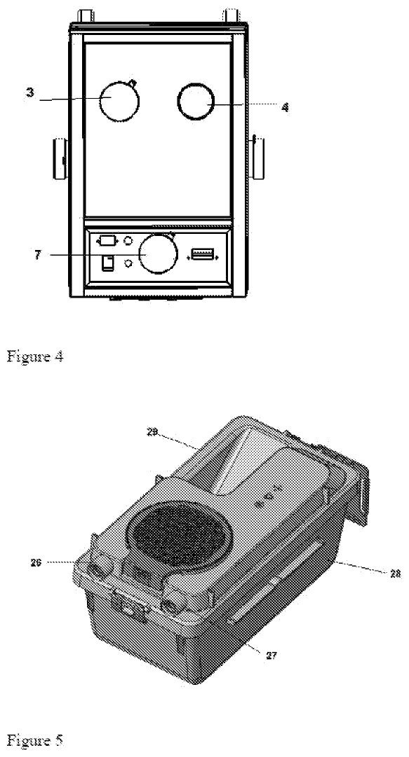

Each IVC Cage 11 (FIG. 5) has one Cage air supply valve 27 and Cage air exhaust valve 26 situated either on Cage bottom 28 or Cage lid 29. Air from the Rack for holding IVC Cage 10, passes through the Supply distribution network 8 to the Cage air supply valve 27 of the IVC Cage 11. Filtered air then reaches inside the IVC Cage 11 providing ventilation for the animals within the IVC Cage 11.

Air inside the IVC Cage 11 is then exhausted from the Cage air exhaust valve 26 using the Exhaust distribution network 9 to the Exhaust port 5 and or 6 of the Ventilator 1 due to the suction created by Exhaust blower or fan 16. The exhausted air first reaches to initial exhaust chamber 22 fitted with one or more UV-C light 19. The UV-C light 19 may be fitted before or after the filter or between pre-filter 18 and HEPA filter or ULPA filter 17 or at any two locations mentioned above or at all the three locations mentioned herein. The wattage of UV-C 19 light is selected to provide complete disinfection of the volume of air that is being exhausted and within its residence time in the air exhaust side. The UV-C light 19 acts as germicidal lamp and kills the bacteria, viruses, mold and other pathogens in the stream of the air that is taken back from the cage and rack system.. UV-C light 19 acts as a germicidal lamp killing the bacteria, viruses, mold and other pathogens in the exhausted air taken back from the cage. This air already treated with UV-C light 19 then passes through Exhaust Pre-filter 18 and Exhaust HEPA or ULPA filter 17 for further filtration. The treatment of air by UV-C light 19 can be either before or after filtration or at both locations This filtered exhausted air then passes through the duct of Exhaust blower 24, to fan 16 and exhaust port 7 and then eventually released to the external environment by suitable means. The UV C light can be fitted at any position below or above or at both locations with respect to HEPA/ULPA filter/s of the exhaust section.

Ventilator unit 1 has control panel 2 where different parameters like air changes per hour (ACPH), positive or negative pressure modes and number of cages can be set as well as many different parameters like temperature, relative humidity, ammonia etc. can be monitored and displayed.

### REFERENCES

1. https://www.light-sources.com/blog/killing-bacteria-with-uv-light/
2. https://www.light-sources.com/blog/killing-bacteria-with-uv-light/
3. https://www.phonesoap.com/pages/faq-uv-c-light-technology
4. Chun-Chieh Tseng and Chih-Shan Li, Inactivation of Virus-Containing Aerosols by UltravioletGermicidal Irradiation, Aerosol Science and Technology, 39:1136-1142, 2005
5. Bintsis T, Litopoulou-Tzanetaki E, Robinson RK, 2000. Existing and potential applications of ultraviolet light in the food industry—a critical review. J Sci Food Agric80: 637-645. doi:10. 1002/(SICI)1097-0010(20000501)80: 637::AID-JSFA603>3.0.CO;2-1.
6. Brian R. Yaun, Susan S. Sumner Joseph D. Eifert Joseph E. Marcy, Inhibition of pathogens on fresh produce by ultraviolet energy, International Journal of Food Microbiology, Volume 90, Issue 1, 1 Jan. 2004, Pages 1-8
7. Do-Kyun Kim, Dong-Hyun Kang, UVC LED Irradiation Effectively Inactivates Aerosolized Viruses, Bacteria, and Fungi in a Chamber-Type Air Disinfection System, September 2018, Volume 84, Issue 17, e00944-18.
8. Lyndon I. Larcomand Nitin H. Thaker, The effects of temperature and ultraviolet irradiation on multiplication of bacteriophage, Biophysical Journal, Volume 19, 1977, 029
9. Chris Walker, Gwangpyo Ko, Effect of Ultraviolet Germicidal Irradiation on Viral Aerosols, Environmental Science and Technology, 41(15):5460-5 Sep. 2007.

1. Individually Ventilated Caging system (IVC system) for small animals comprising a Ventilation Unit 1 comprising an initial supply chamber 12 for taking external air inside the system for filtering and supplying to the cages in IVC system and an exhaust chamber 22 for exhausting the used air collected from the cages in the IVC system; the supply chamber and the exhaust chamber provided with filter/s 13, 14, 17, 18 for the incoming or outgoing/exhausting air; wherein the IVC system is characterized by providing devices additional to the filters for disinfecting incoming air and/or disinfecting out-going air exhausted from the cages.

2. The IVC system as claimed in claim 1 wherein the devices additional to the filters for disinfecting incoming air and/or disinfecting out-going air exhausted from the cages comprises:
a. UV C light 15, and/or
b. one or more electrostatic air filter/s and/or
c. an ionizer on supply and or exhaust side.

3. The IVC system as claimed in claim 1, comprising:
a. Ventilator unit 1 comprising:
i. a supply section and/or an exhaust section,
ii. HEPA or ULPA filters provided in supply section and exhaust section for filtering air passing through these sections,
iii. UV-C light 15 to treat filtered air through supply ports 3 and 4 to the IVC Cages placed on the Rack 10 through a supply distribution network 8 connected to ventilator unit 1 through a suitable flexible or non flexible pipe 30a,
b. an exhaust distribution network 9 of the rack for collecting and return of the used air from the cages back to the Ventilator unit 1 through an exhaust port 6 of the Ventilator Unit 1, the exhaust distribution network 9, the exhaust port being interconnected with the exhaust distribution network 9 through a flexible or non flexible pipe 30b, the exhaust air brought back to Ventilation Unit 1 being treated with UVC light 19 and being filtered through HEPA- or ULPA-filtration; and return to the external environment though exhaust port 7 of the Ventilator unit 1.
c. the Ventilator unit 1 comprising a Control Panel with display 2 where different operational parameters are displayed.

4. The IVC system as claimed in claim 3, wherein,
a. one or more Racks 10 for holding IVC Cages are provided each being supplied with the UV-C light 15 treated filtered air through additional supply ports,
b. parameters displayed on the Control Panel consist of one or more selected from the group consisting of air changes per hour, positive or negative pressure modes, number of cages, temperature, relative humidity, ammonia and other environmental parameters.

5. The IVC system as claimed in claim 1, wherein:
a. the Ventilator unit 1 has upper half as a supply section and lower half as exhaust section.
i. the supply section comprises one or more supply blower or fan 12, duct of supply blower 23, supply side HEPA or supply side ULPA filter 13, supply side pre-filter 14, one or more UV-C lights 15, initial supply chamber 21 and final supply chamber 25.
ii. the exhaust section comprises one or more Exhaust blower or fan 16, Duct of exhaust blower 24, exhaust side HEPA or exhaust side ULPA filter 17, exhaust side pre-filter 18, one or more UV-C light 19 and initial exhaust chamber 22,
b. the Ventilator unit 1 has:
i. filtered air supply ports on supply side labeled as supply port no. 3 and 4, each one connecting to separate rack units 10 for holding the IVC Cage in the supply distribution network 8; and
ii. exhaust air outlet ports on exhaust side labeled as exhaust port no. 5 and 6 coming from two separate rack units 10 for connecting with the Exhaust Distribution Network 9 of one or more rack for holding the IVC Cages 11.

6. The IVC system as claimed in claim 1, wherein:
a. the Ventilator unit 1 comprises an air inlet 20 for taking external air in for filtration and circulation,
b. supply blower or fan 12 takes the air from the external environment through the air inlet 20 for filtration, treatment with UV-C light 15 and supplies to the IVC Cages,
c. the initial supply chamber 21 is fitted with one or more UV-C light 15 either above the filters or below the prefilter or between pre-filter 14 and HEPA filter or ULPA filter 13 or at any two locations mentioned above or at all the three locations mentioned herein,
d. the Ventilator unit 1 comprises an air supply port on supply side as supply port nos. 3 and 4 for connecting with the Rack 10 that holds the IVC Cages
e. the initial exhaust chamber 22 is fitted with one or more UV-C light 19. The UV-C light 19 may be fitted before or after the filter or between pre-filter 18 and HEPA filter or ULPA filter 17 or at any two locations mentioned above or at all the three locations mentioned herein.
f. in the initial exhaust chamber 22 the air is passed through supply Pre-filter 14 and supply side HEPA or supply side ULPA filter 13 for further removing the particles in the air stream,
g. this filtered air then passes through the duct of supply blower 23 and through the supply blower or fan 12 and then to the final supply chamber 25,
h. filtered air in the final supply chamber 25 then passes through the supply ports 3 and or 4 or more ports in case more Racks are connected to the Ventilator Unit 1 to the supply distribution network 8 of the Rack 10 for holding IVC Cage and eventually to the IVC Cages 11 loaded on the Rack 10,
i. the air from the cage is exhausted, filtered and treated with UV-C light in the exhaust section of the Ventilator unit 1 for disinfection and the treated air is released to the external environment from the outlet port 7.

7. The IVC system as claimed in claim 6 wherein the wattage of UV-C 15 and UV-C 19 lights is selected to provide disinfection of the volume of air that is drawn in respective supply chamber and exhaust chamber and within residence time of the air within these chambers.

8. The IVC system as claimed in claim 1, wherein: each IVC Cage 11 has one Cage air supply valve 27 and/or Cage air exhaust valve 26 situated either on Cage bottom 28 or alternatively they can also be situated on Cage lid 29. Cage lid 29 and cage bottom 28 are connected together so as to form airtight connection:
a. air from the Rack for holding IVC Cage 10, passes through the supply distribution network 8 to the Cage air supply valve 27 of the IVC Cage 11,
b. filtered and UV-C light treated air then reaches inside the IVC Cage 11 providing ventilation for the animals within the IVC Cage 11.
c. air inside the IVC Cage 11 is then exhausted from the Cage air exhaust valve 26 using the Exhaust distribution network 9 to the Exhaust port 5 and or 6 of the Ventilator 1 due to the suction created by Exhaust blower or fan 16,
d. the exhausted air first reaches for treatment and filtration to initial exhaust chamber 22 fitted with one or more UV-C light 19, pre-filter 18 and exhaust side HEPA or exhaust side ULPA filter 17,
e. this air after treatment with UV-C light 19 and filters, then passes through the duct of exhaust blower 24, to fan 16 and exhaust port 7 and then eventually released to the external environment by suitable means.

9. The IVC system as claimed in claim 1, wherein the small animals consist of mice and guinea pigs.
